(19) United States
(12) Patent Application Publication
(10) Pub. No.: US 2016/0158440 A1
(43) Pub. Date: Jun. 9, 2016

(54) Simplified Microplegia Delivery System

(71) Applicant: april radicella, marietta, GA (US)

(72) Inventor: april radicella, marietta, GA (US)

(21) Appl. No.: 15/046,432

(22) Filed: Feb. 17, 2016

Related U.S. Application Data
(63) Continuation-in-part of application No. 14/091,540, filed on Nov. 27, 2013.

(51) Int. Cl. A61M 5/168; A61M 5/172; G06F 19/00; A61M 5/145

(52) U.S. Cl. CPC A61M 5/16831; A61M 5/1452; A61M 5/172; G06F 19/3468; A61M 2205/505; A61M 2005/14208

(57) ABSTRACT

Cardioplegia is a mixture of blood and cardioplegic solution administered through the coronary vessels to protect the myocardium during cardiopulmonary bypass procedures. Microplegia describes the use of concentrated cardioplegic solution during cardioplegia delivery in an effort to reduce hemodilution and improve myocardial protection. This new apparatus is designed to deliver concentrated cardioplegic solution during cardioplegia delivery. The apparatus features cost saving opportunities, adaptability to any cardioplegia system, simplified delivery options, and safety options. Cardioplegia flow sensor integration to the apparatus allows for convenient auto start/stop as well continuously updated flow-based delivery of the cardioplegic solution during cardioplegia delivery.

### FIELD OF INVENTION

The invention generally relates to the field of cardiovascular surgical procedures requiring administration of cardioplegia.

### BACKGROUND OF THE INVENTION

Cardiovascular perfusionists play a unique role during cardiovascular surgical procedures; one goal is to create a bloodless, motionless field while the cardiac surgeon performs repairs.

To achieve a bloodless field, systemic blood flow from the vena cava is diverted to the heart/lung machine FIG. 1 (Prior Art) and returned to the aorta thus bypassing the heart and lungs.

To create a motionless field, perfusionists administer cardioplegia (1). From the Greek, cardioplegia translates to “paralysis of the heart;” its purpose is to protect, preserve, and quiet the myocardium. Turning attention to FIG. 2 (Prior Art), cardioplegia delivery involves diverting a portion of oxygenated blood from the systemic pump (2), adding a cardioplegic solution (3), and pumping (4) that mixture to the myocardium of the patient.

For the purpose of this explanation, the term “cardioplegia” refers to the variable blood/cardioplegic solution mix periodically delivered to the heart for myocardial protection, and the term “cardioplegic solution” refers to the arresting agent diluted in crystalloid to various concentrations depending on the blood to cardioplegic solution delivery ratio and surgeon preference.

Potassium is the effective drug used in cardioplegic solutions. A high potassium dose prevents repolarization causing electrical arrest; electrical arrest leads to mechanical arrest thus decreasing myocardial oxygen demand.

⁶Currently, cardioplegia may be given at various blood to cardioplegic solution ratios ranging from full crystalloid, 4:1, 8:1, 16:1, to microplegia. The initial cardioplegia dose contains a high dose of potassium; the potassium level is titrated down as the surgery proceeds. Mirroring the potassium levels in the cardioplegia, blood serum potassium levels rise after the induced cardiac arrest and gradually return to normal.

Microplegia is a term used to refer to a cardioplegic solution delivery system that uses minimal amounts of crystalloid to carry the potassium to the heart. Perfusionists are continually developing techniques to decrease the amount of crystalloid used during cardiovascular surgical procedures in an effort to improve patient outcomes and decrease blood transfusion rates.

To put it in perspective, an example cardiac procedure, using 4:1 cardioplegia, will deliver anywhere from 200 ml to 1500 ml of cardioplegic solution (contributing to hemodilution). In contrast, a microplegia system in the same procedure will deliver 5 ml to 40 ml of cardioplegic solution.

One option to deliver microplegia is to manipulate a syringe driver; this technique is effective, but it requires extensive calculations and can be difficult to use during a cardiopulmonary bypass run. I have designed a simplified, cost-effective syringe driver with standardized and automatically updating flow calculations.

### COMPARABLE ITEM ON THE MARKET

Dollar et. al, U.S. application Ser. No. 13/613,705 apparatus termed “MPS” includes a complete cardioplegia delivery system, dispensing cardioplegic solution, pumping blood pulled from the oxygenator, a heat exchanger to control the temperature of the blood/cardioplegic solution mix, various pressure gauges, and air detectors. This apparatus does not utilize a syringe driver to dispense cardioplegic solution; instead, it uses a series of piston pumps to depress fluid pockets filled with cardioplegic solution. There are two complaints/problems with this apparatus: 1) The MPS and its components can be expensive, 2) The MPS is difficult and time consuming to override in the event of a failure.

U.S. application Ser. No. 13/613,705 is a stand-alone product, whereas the Simplified Microplegia Delivery System is used in conjunction with various current cardioplegia delivery systems utilizing their blood delivery consumable, heat exchanger, and pressure transducer. This makes the Simplified Microplegia Delivery System adaptable to current surgeon and perfusion preferences, lowers the cost of consumables, and offers unique override abilities in the event of a device failure. The unique, continuous flow-calculating feature communicated via the flow sensor simplifies the delivery process making this accessible and practical for all clinicians.

### SUMMARY OF THE INVENTION

The Simplified Microplegia Delivery System (SMDS) is a syringe driver apparatus FIG. 3 that delivers cardioplegic solution during cardiopulmonary bypass surgical procedures. The apparatus uses a mechanical arm (5) to depress an upright, gripped (6) syringe at a rate determined by the clinician as described in the features list.

Features:

1.) Adaptability.

The SMDS adapts to any cardioplegia delivery system. The SMDS replaces the highly diluted cardioplegic solution bags in any cardioplegia delivery system (3). The cardioplegic solution is integrated into the cardioplegia delivery line (7) by cutting in a 3/16 luer lock straight connector (8) distal to the cardioplegia pump (4).

2.) Auto start/stop.

Cardioplegic solution delivery via the SMDS initiation and termination corresponds with the cardioplegia delivery initiation and termination as communicated via the adjustable flow sensor (9). Clinicians will appreciate this easy start and stop feature considering that it eliminates steps in the cardioplegia delivery process.

3.) Simplified delivery options.

Turning attention to FIG. 4, the clinician has the option: to deliver cardioplegic solution at a high potassium flow rate (10), to deliver cardioplegic solution at a low potassium flow rate (11), to deliver a bolus of cardioplegic solution (12), and finally, to cease cardioplegic solution delivery (13). When cardioplegic solution delivery is ceased, or in standby, the device will continue to count delivery volumes of the cardioplegia as communicated via the flow sensor. The delivery options are limited for ease of use and mimics terminology that clinicians are accustomed to hearing.

4.) Preset flow-based calculations.

Cardioplegia flow rates are continuously communicated to the SMDS via the flow sensor. The SMDS software uses this input to calculate the appropriate cardioplegic solution dispense-rate under the clinician's designated delivery selection described in. Number 3 of the Features section.

5.) Customization.

Standard, pre-set flow options are customizable, within the deliverable range, in order to meet institute, clinician, and/or surgeon preference. Mann volume and display are customizable. Customizations are made using the program keys (14).

6.) Display.

The displays shows various pertinent clinical information comprising the flow rate of the cardioplegia in mL/min (15), the flow rate of the cardioplegic solution in mEq/L (16), the total delivery volume of the cardioplegia in mL (17), the total current delivery volume of cardioplegia in mL (18), and a timer which counts active delivery (19) and time between cardioplegia doses (20). The display also houses an indicator for the battery (21), and alert/alarm information.

7.) Cost.

The SMDS enables clinicians to deliver concentrated cardioplegic solution, naturally reducing hemodilution. The simple potassium concentrate required, reduces costly pharmaceutical solutions. There is minimal, cost of disposables associated with use of the SMDS; apparatus is designed to operate with various current syringe designs, male/female pressure lines, and a 3/16^{th} straight connector with luer lock.

8.) Safety.

Because it is adaptable to any cardioplegia delivery system, the clinician has the option to easily revert to the previous cardioplegia system; this feature allows the clinician to meet surgeon preference, as well as providing an immediate backup option in the event of a device failure. Additionally, the device has an internal, rechargeable battery with indicator symbols on the front screen. A corresponding indicator is illuminated to confirm the active delivery mode (22), reducing clinician errors. A visual and audible alert is associated with the time count between cardioplegia doses, as specified by the clinician. A visual and audible alarm is associated with the battery supply. All alerts and alarms can be silenced (23).

¹³Considering the rising costs and risks of with blood transfusions associated with hemodilution during cardiopulmonary bypass, there is an immense need for an inexpensive, user-friendly microplegia system. This apparatus simplifies complicated delivery calculations, finally making microplegia an affordable and safe option for institutions.

### BRIEF DESCRIPTION OF THE DRAWINGS

FIG. 1 (Prior Art) is a diagram of the heart lung machine.

FIG. 2 (Prior Art) is a diagram of a typical cardioplegia delivery set up.

FIG. 3. is a diagram of a typical cardioplegia set up with the SMDS incorporated.

FIG. 4. shows the display of the apparatus.

### DETAILED DESCRIPTION OF THE DRAWINGS

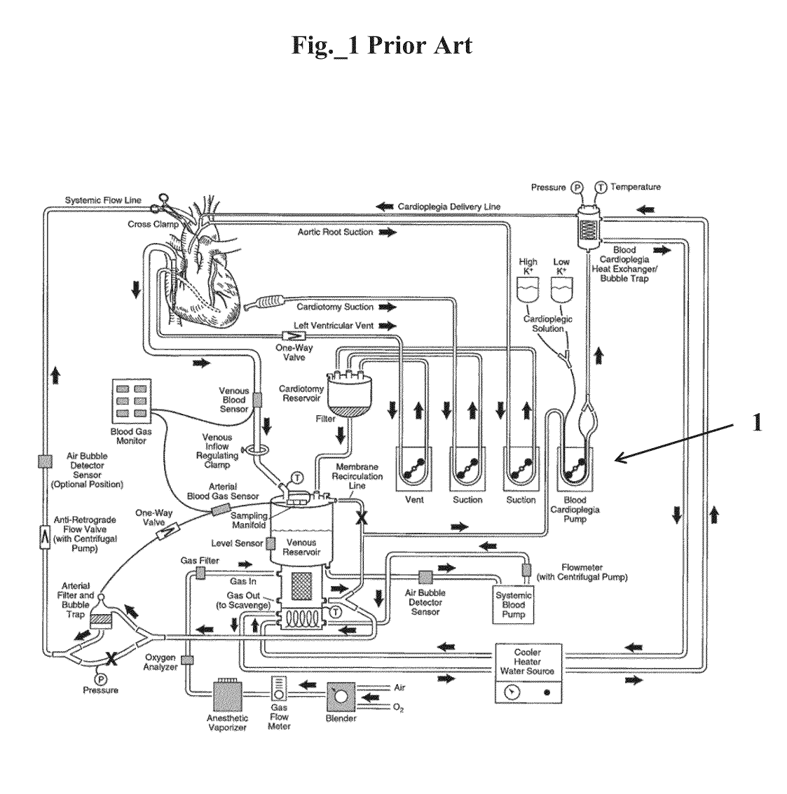

FIG. 1 (Prior Art) is a detailed diagram of the heart lung machine, showing the general components of the heart lung machine, as well as the path of blood as it travels from the vena cava through the heart lung machine and returns to the aorta. A portion from the oxygenated blood is diverted to the cardioplegia system (1) for myocardial delivery.

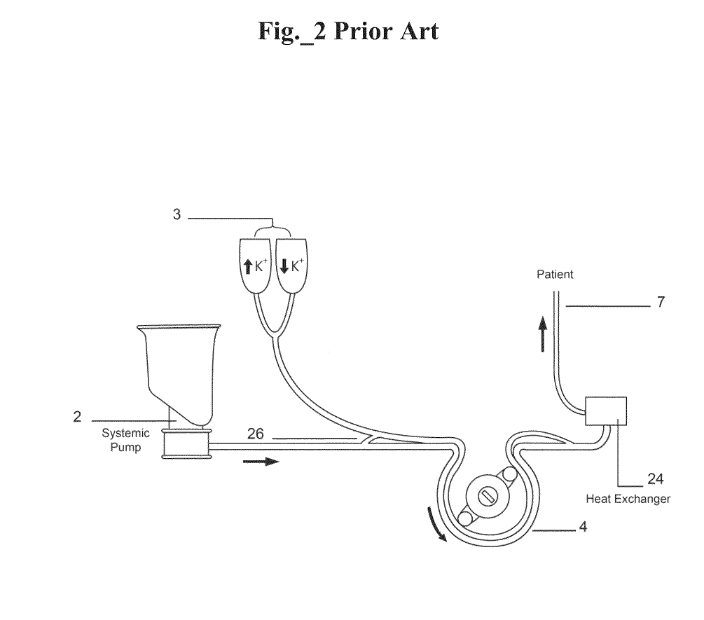

FIG. 2 (Prior Art) is a drawing demonstrating the cardioplegia circuit. Blood is pulled from the oxygenator of the systemic pump (2), to the roller pump (4), through the heat exchanger (24), to the myocardium. Two bags of standard cardioplegic solutions (high potassium and low potassium concentrations) are shown (3). Cardioplegic solution is mixed into the blood post pump (4).

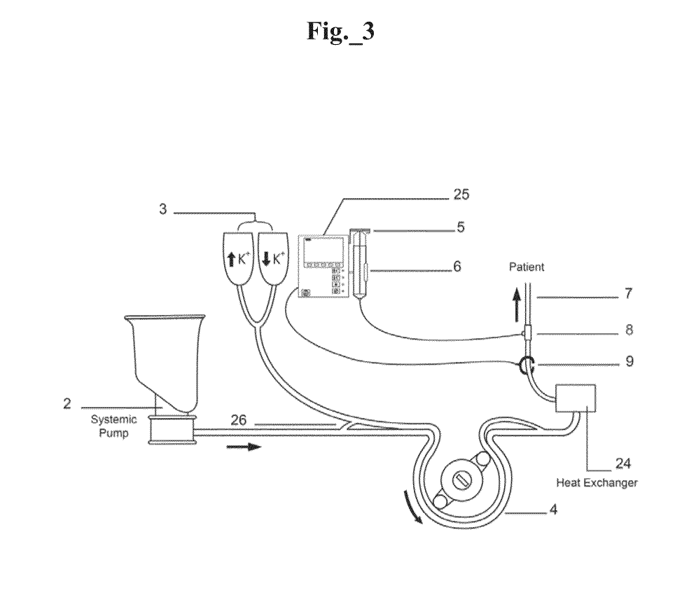

FIG. 3 shows the incorporation of the SMDS (25) with a typical cardioplegia set up. No volume is pulled from the dilute cardioplegic solutions (3); instead, only blood is pulled through the pump by way of a bypass line (26). The apparatus (25) uses a mechanical arm (5) to depress an upright, gripped (6) syringe. The cardioplegic solution is integrated into the cardioplegia circuit by way of a 3/16^{th} straight luer lock connector (8). The flow sensor (9) attaches to the delivery line (7) to transmit blood flow to the SMDS for flow calculations.

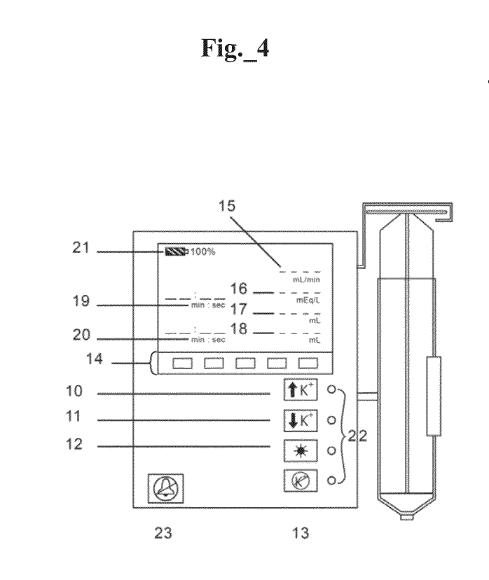

FIG. 4 demonstrates the front of apparatus. There are four delivery options (10-13) with corresponding indicator lights (22) as a visual means to confirm delivery mode to the user. The screen shows pertinent information (15-21). There are various programming keys (14) for programming. There is a silence key (23) available to conveniently silence alerts and alarms.

1. An apparatus for the delivery of cardioplegic solution comprising,
i.) a syringe driver,
wherein there are three cardioplegic solution delivery mode options: high dose, low dose, and bolus;
wherein there is one standby mode, where no cardioplegic solution is delivered, but the device continues to collect and display data.
ii.) a customizable front display panel comprising a touch screen displaying cardioplegic solution delivery information,
wherein display options comprise the flow rate of the cardioplegia solution, the total delivery volume of cardioplegia, the total current delivery volume of cardioplegia, a timer counting active delivery, a timer counting the time between cardioplegia doses, corresponding audible and visual alarms, alarm silence, touch program keys,
iii.) an adaptable flow sensor
wherein the flow sensor is configured to transmit the cardioplegia flow to the apparatus;
wherein the apparatus' software uses the transmitted flow measurements to calculate and initiate/terminate the corresponding cardioplegic solution dispense rate according to the designated delivery mode;
wherein the pre-set flow based calculations are customizable according to institute preference within the deliverable range.
